(19) United States
(12) Patent Application Publication
(10) Pub. No.: US 2021/0214325 A1
(43) Pub. Date: Jul. 15, 2021

(54) PRODUCTION OF PROTOANEMONIN BY OXIDATION OF LEVULINIC ACID OR ANGELICALACTONE ISOMERS

(71) Applicants: Jesse Quentin Bond, Syracuse, NY (US); Ran Zhu, Boston, MA (US)

(72) Inventors: Jesse Quentin Bond, Syracuse, NY (US); Ran Zhu, Boston, MA (US)

(73) Assignee: SYRACUSE UNIVERSITY, SYRACUSE, NY (US)

(21) Appl. No.: 17/148,615

(22) Filed: Jan. 14, 2021

Related U.S. Application Data
(60) Provisional application No. 62/960,848, filed on Jan. 14, 2020.

(51) Int. Cl. C07D 307/20; B01J 23/22

(52) U.S. Cl. CPC C07D 307/20; B01J 23/22

(57) ABSTRACT

A process for forming protoanemonin from an amount of levulinic acid or an amount of α-Angelica lactone using oxidative dehydrogenation. Biomass-derived levulinic acid (LA) is a green platform chemical and, using an oxidative scission pathway can be transformed into cyclic intermediates, namely angelicalactones to form protoanemonin. The oxidative dehydrogenation may be heterogeneously catalyzed in a gas-phase to perform aerobic oxidation using a solid oxide such as vanadium oxide. Protoanemonin is an intriguing polyfunctional molecule that is uniquely suited to bio-based production, and can be synthesized in yields from 50%-75% during periods of transient reactor operation.

### CROSS-REFERENCE TO RELATED APPLICATIONS

This application claims priority to U.S. Provisional Application No. 62/960,848, filed on Jan. 14, 2020.

### STATEMENT REGARDING FEDERALLY SPONSORED RESEARCH OR DEVELOPMENT

This invention was made with government support under Grant No. 1454346 awarded by the National Science Foundation (NSF). The government has certain rights in the invention.

### BACKGROUND OF THE INVENTION

### 1. Field of the Invention

The present invention relates to the synthesis of protoanemonin and, more specifically, to a process for the production of protoanemonin using a solid oxide catalyst and a gas phase process.

### 2. Description of the Related Art

Protoanemonin is a polyfunctional molecule having a lactone ring structure coupled with a conjugated diene, which makes it interesting as a polymer precursor. There has also been historical interest in protoanemonin's antimicrobial properties within the medical research community. It has previously been synthesized using conventional wet chemistry methods that would be difficult to implement at commodity scale. Accordingly, there is a need in the art for an approach for producing protoanemonin that can be used at large scale.

### BRIEF SUMMARY OF THE INVENTION

The present invention comprises an approach for converting bio-based levulinic acid and its dehydrated analogs (alpha-angelicalactone, alpha'-angelicalactone, and beta-angelicalactone) into 5-methylene-2(5H)-furanone, also known by its common name, protoanemonin. The core of the technology is oxidative dehydrogenation of β-angelicalactone over reducible oxides (e.g., Vanadium, Cerium, Titanium, etc), which forms protoanemonin. If starting with alpha isomers of angelicalactones, supported solid oxides will also facilitate isomerization to form β-angelicalactone under the same conditions; as such, they can be used as feedstocks. Analogously, solid oxides also facilitate levulinic acid dehydration to form alpha angelicalactone isomers, and so it can also be used as a starting material.

The present invention is thus a process of forming protoanemonin, comprising the steps of providing an amount of levulinic acid or an amount of α, α′, or β-Angelica lactone and performing oxidative dehydrogenation of the amount of levulinic acid or the amount of α, α′, or β-Angelica lactone to form an amount of protoanemonin. The step of oxidative dehydrogenation may be heterogeneously catalyzed. The step of oxidative dehydrogenation may be performed in a gas-phase. The step of oxidative dehydrogenation may comprise aerobic oxidation. The step of oxidative dehydrogenation may be performed with a solid oxide catalyst. The solid oxide catalyst may be vanadium oxide. The step of oxidative dehydrogenation may include the formation of β-Angelica lactone as an intermediate product. The step of oxidative dehydrogenation may be performed at a temperature of 473 Kelvin. Thus, the present invention employs a simple solid oxide catalyst and a gas phase process that can use air as the oxidant and operate in continuous flow. Observed yields in the range of 50-75% were achieved upon initial optimization, suggesting that the present invention may be scalable.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a scheme for the oxidation of levulinic acid to form maleic anhydride;

FIG. 2 is a scheme for the methyl and alkyl oxidative scission pathways for methylketones;

FIG. 3 is a graph of the rates of oxidative scission of 2-propanone and 2-propanone-d6. (470 K, P2P=12.3 Torr, PO2=133 Ton, He Balance);

FIG. 4 is a schematic of open chain (enol-mediated) and cyclic (lactone-mediated) pathways for the oxidative scission of levulinic acid;

FIG. 5 is a graph of the FTIR spectra of 2-pentanone (2-P), levulinic acid (LA), α-angelicalactone (α-AL), and maleic anhydride (MA) over VOₓ/γ-Al2O3 at a) T=473K; b) the zoomed in version of C-13 H stretching region at various reaction temperature, P_{Probe molecule}=0.2 Torr, P_{O2}=133 Torr, He balance;

FIG. 6 is a graph of a) ¹³C-NMR spectrum and b)¹H-NMR spectrum for a representative a product mixture obtained during the oxidation of levulinic acid/α-angelicalactone. Samples dissolved in C₃D₆O;

FIG. 7 is a diagram of the Chemical shifts in ¹³C-NMR for major products from the oxidation of levulinic acid. Predicted values are given in a different shade;

FIG. 8 is a graph of the conversion of α-angelicalactone and corresponding yields to protoanemonin and maleic anhydride over VOₓ/γ-Al₂O₃. PR=8 Torr, P_{O2}=133 Ton, T=528K;

FIG. 9 is a graph of the carbon selectivity during the oxidation of a) levulinic acid, b) α-angelicalactone on VOₓ/γ-Al₂O₃. T=550 K, P_{probe molecule}=5 Torr, P_{O2}=133 Torr, He balance;

FIG. 10 is a scheme for maleic anhydride formation during the oxidation of levulinic acid;

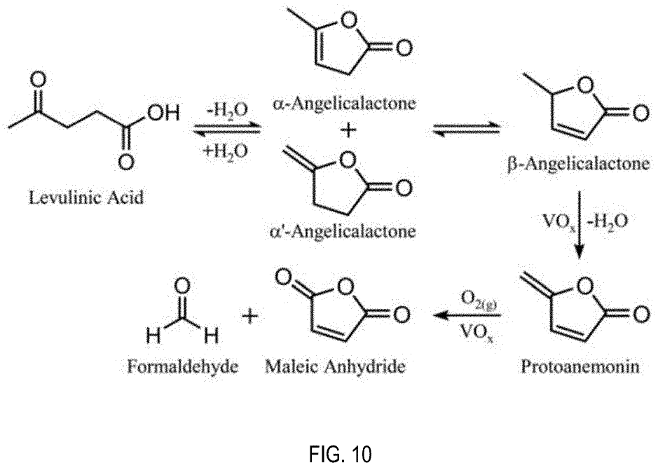

FIG. 11 is a graph of ¹³C NMR for mixed reaction products dissolved in 2-propanone-d6 (C₃D₆O)

FIG. 12 is a graph of the ³C spectrum for a mixture of reaction products obtained by bubbling the reactor effluent gas through deuterated acetone (C₃D₆O) under conditions of optimal selectivity toward the targeted unknown compound (96 m/z according to GCMS); this sample includes an externally introduced acetic acid standard, permitting independent resolution of peaks associated with acetic acid;

FIG. 13 is a graph of the ³C spectrum for a mixture of reaction products obtained by bubbling the reactor effluent gas through deuterated acetone (C₃D₆O) under conditions of optimal selectivity toward the targeted unknown compound (96 m/z according to GCMS); this sample includes an externally introduced α-angelicalactone standard, permitting independent resolution of its associated peaks;

FIG. 14 is a graph of the ³C spectrum for a mixture of reaction products obtained by bubbling the reactor effluent gas through deuterated acetone (C₃D₆O) under conditions of optimal selectivity toward the targeted unknown compound (96 m/z according to GCMS); this sample includes an externally introduced maleic anhydride standard, permitting independent resolution of its associated peaks;

FIG. 15 is a graph of the ¹H spectrum for a mixture of reaction products obtained by bubbling the reactor effluent gas through deuterated acetone (C₃D₆O ) under conditions of optimal selectivity toward the targeted unknown compound (96 m/z according to GCMS); however, they include contributions from other reaction products as it was not possible to operate at 100% yield, nor was it possible to easily isolate the unknown subsequent to collection;

FIG. 16 is a graph of the ¹H spectrum for a mixture of reaction products obtained by bubbling the reactor effluent gas through deuterated acetone (C₃D₆O ) under conditions of optimal selectivity toward the targeted unknown compound (96 m/z according to GCMS); this sample includes an externally introduced acetic acid standard, permitting independent resolution of its associated peaks

FIG. 17 is a graph of the ¹H spectrum for a mixture of reaction products obtained by bubbling the reactor effluent gas through deuterated acetone (C₃D₆O ) under conditions of optimal selectivity toward the targeted unknown compound (96 m/z according to GCMS); this sample includes an externally introduced α-angelicalactone standard, permitting independent resolution of its associated peaks;

FIG. 18 is a graph of the ¹H spectrum for a mixture of reaction products obtained by bubbling the reactor effluent gas through deuterated acetone (C₃D₆O ) under conditions of optimal selectivity toward the targeted unknown compound (96 m/z according to GCMS); this sample includes an externally introduced maleic anhydride standard, permitting independent resolution of its associated peaks;

FIG. 19 is a graph of the ¹H—¹³C HSQC spectrum for a mixture of reaction products obtained by bubbling the reactor effluent gas through deuterated acetone (C₃D₆O ) under conditions of optimal selectivity toward the targeted unknown compound (96 m/z according to GCMS);

FIG. 20 is a graph of FTIR spectra of calcined γ-Al₂O₃ at various reaction temperatures. P_{O2}=133 Torr and He balanced;

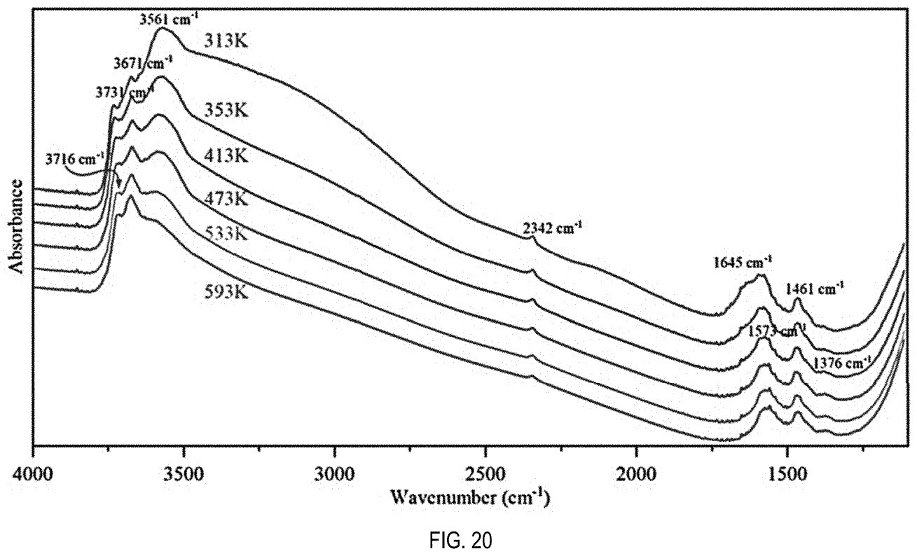

FIG. 21 is a graph of FTIR spectra of calcined VOₓ/γ-Al₂O₃ (8 V/nm²) at various temperatures. P_{O2}=133 Torr and He balanced;

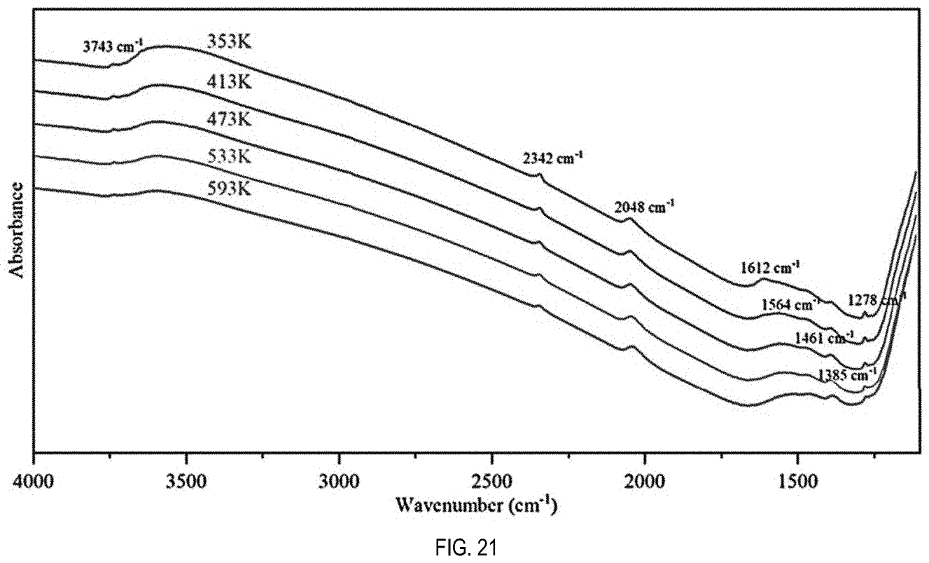

FIG. 22 is a graph of FTIR spectra of VOₓ/y-Al₂O₃ (8 V/nm²) exposed to 0.2 Torr α-pentanone during steady-state oxidation at various temperatures. P_{O2}=133 Torr and He balanced;

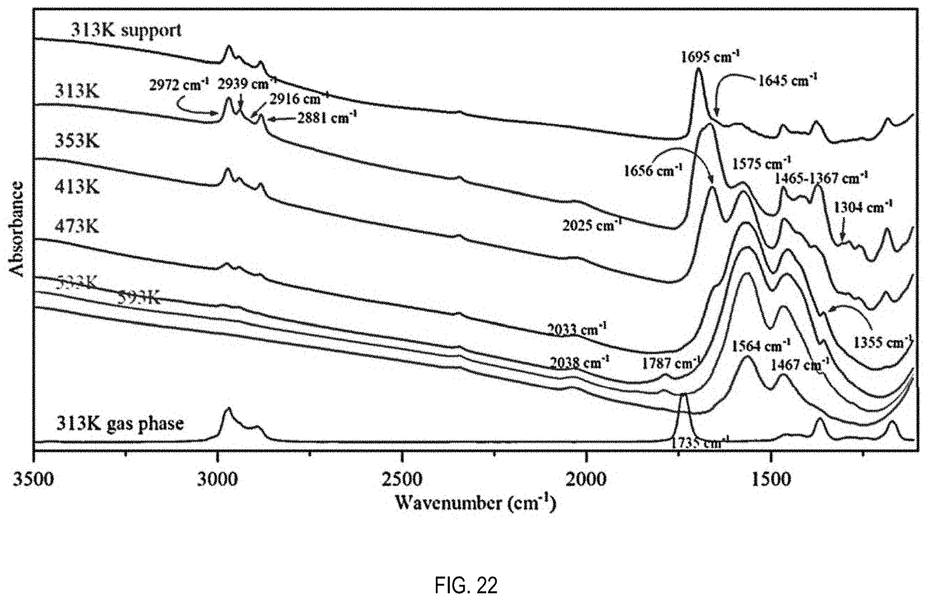

FIG. 23 is a graph of FTIR spectra of VOₓ/γ-Al₂O₃ (8 V/nm²) exposed to 0.2 Torr levulinic acid during steady-state oxidation at various temperatures. P_{O2}=133 Torr and He balanced;

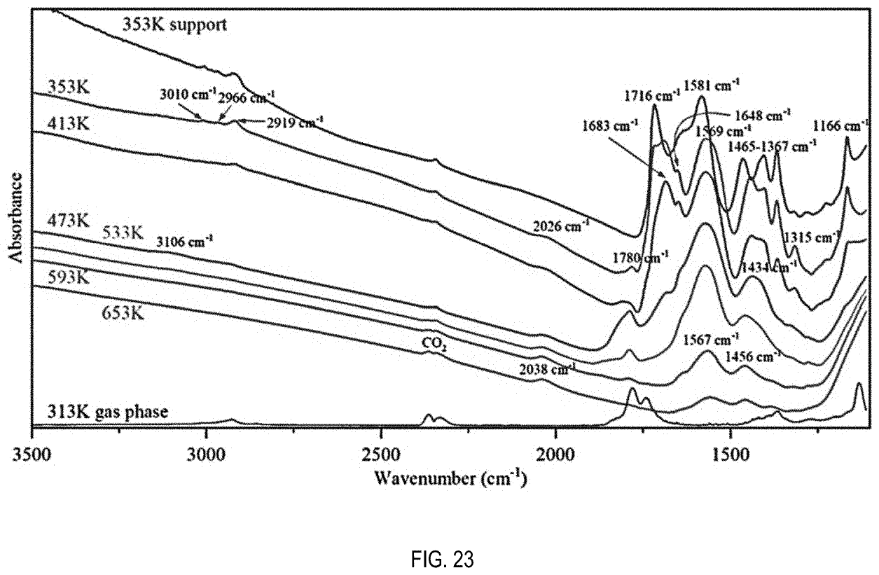

FIG. 24 is a graph of FTIR spectra of VOₓ/γ-Al₂O₃ (8 V/nm²) exposed to 0.2 Torr α-angelicalactone during steady-state oxidation at various temperatures. P_{O2}=133 Torr and He balanced

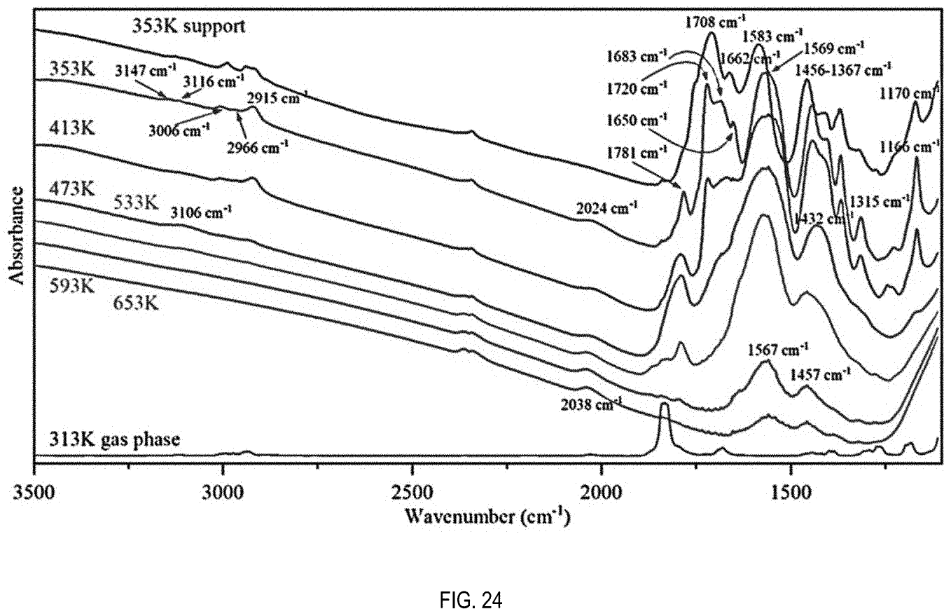

FIG. 25 is a graph of FTIR spectra of VOₓ/γ-Al₂O₃ (8 V/nm²) exposed to 0.15 Torr acetic acid during steady-state oxidation at various temperatures. P_{O2}=133 Torr and He balanced; and

FIG. 26 is graph of FTIR spectra of VOₓ/γ-Al₂O₃ (8 V/nm²) exposed to 0.2 Torr maelic anhydride during steady-state oxidation at various temperatures. P_{O2}=133 Torr and He balanced.

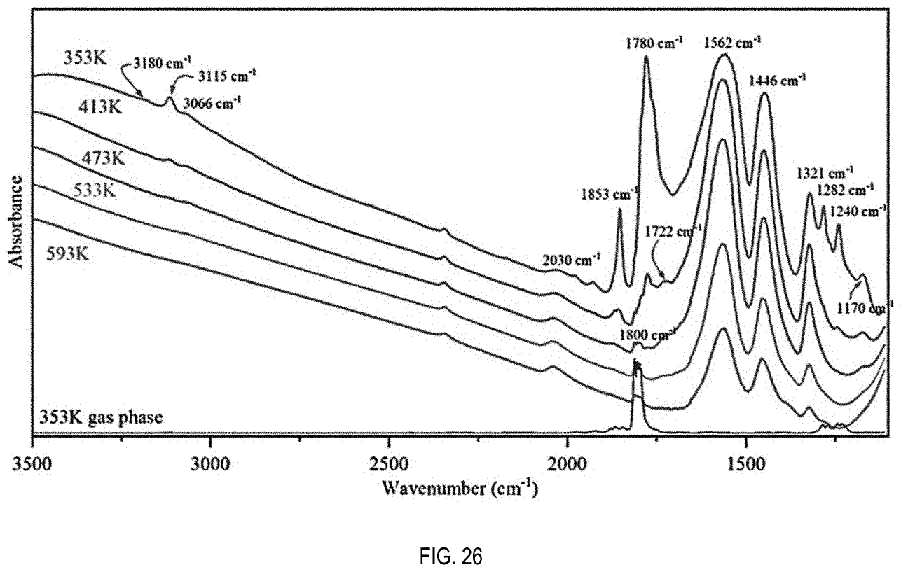

### DETAILED DESCRIPTION OF THE INVENTION

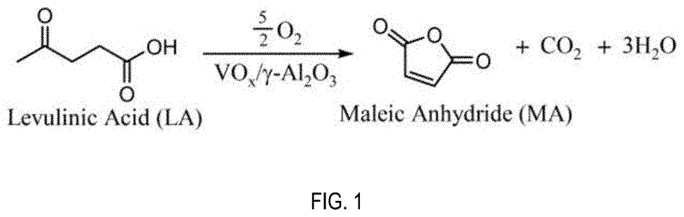

Referring to the figures, wherein like numeral refer to like parts throughout, there is seen in FIG. 1, the vapor-phase, aerobic oxidation of levulinic acid affords maleic anhydride (Scheme 1) in good yield (>70%). The investigation of this unexpected result led to the present invention, which comprises an approach for converting bio-based levulinic acid and its dehydrated analogs (alpha-angelicalactone, alpha′-angelicalactone, and beta-angelicalactone) into 5-methylene-2(5H)-furanone, also known by its common name, protoanemonin. The core of the technology is oxidative dehydrogenation of -angelicalactone over reducible oxides (e.g., Vanadium, Cerium, Titanium, etc), which forms protoanemonin. If starting with alpha isomers of angelicalactones, supported solid oxides will also facilitate isomerization to form (3-angelicalactone under the same conditions; as such, they can be used as feedstocks. Analogously, solid oxides also facilitate levulinic acid dehydration to form alpha angelicalactone isomers, and so it can also be used as a starting material. Thus, the present invention employs a simple solid oxide catalyst and a gas phase process that can use air as the oxidant and operate in continuous flow. Observed yields in the range of 50 - 75% were achieved upon initial optimization, suggesting that the present invention may be scalable

From an oxidative scission perspective, levulinic acid (4-oxopentanoic acid) can be viewed as a methylketone. During oxidation, it can only form maleic anhydride through methyl scission—specifically, though the oxidative scission of the C4-05 bond between the carbonyl carbon and its methyl α-carbon. However, prior accounts of simple methylketone oxidation report only trace selectivity toward methyl scission, which makes a high yield of maleic anhydride noteworthy. The obvious difference between levulinic acid and a methylketone is that levulinic acid is bifunctional, i.e., it is a keto-acid. It is reasonable to expect that the presence of the carboxyl group underlies the stark differences in methyl scission selectivity; however, the mechanistic origins remain unclear. Aerobic ketone oxidation using homogeneous catalysts is well-studied in condensed media, but analogous insights for heterogeneously-catalyzed oxidations are sparse.

One underpinning of the present invention was the resolution of the origin of the unexpectedly high selectivity toward methyl scission during the vapor-phase, oxidation of levulinic acid over vanadium oxides. This was done primarily by comparing the oxidative scission of levulinic acid and various analogs that have been selected to interrogate specific impacts of ketone structure and secondary functionality. Kinetic analysis is supported by FTIR and NMR spectroscopies, which was used to provide insights into the nature of bulk and surface species formed during various oxidation reactions.

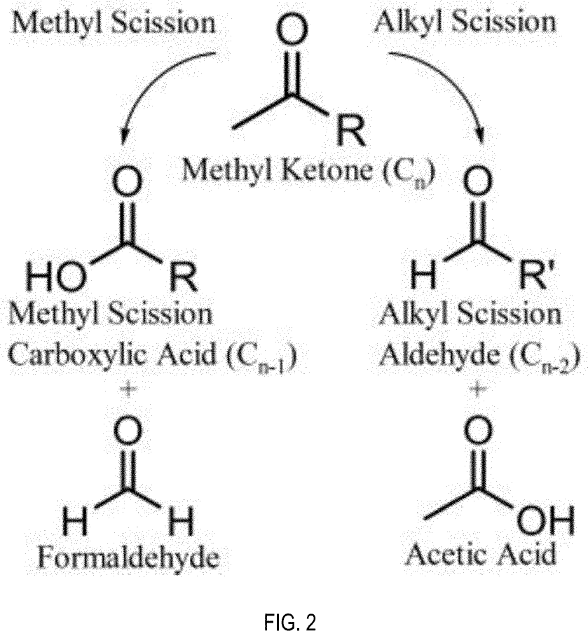

For each probe molecule, rates and selectivities for the oxidative scission of the C—C bonds between the carbonyl carbon and each of its α-carbons are seen in FIG. 2 (Scheme 2). The focus was primarily on asymmetric methylketones, where one α-carbon is a methyl group and the other is an alkyl chain. The two scission pathways are therefore referred to as “methyl scission” and “alkyl scission.” Regardless of the pathway, oxidative ketone scission yields two primary products: a carboxylic acid that is formed by oxidation of the reacting ketone's carbonyl carbon, and an aldehyde or ketone that is formed by oxidation of its cleaved α-carbon. Ketones and aldehydes can undergo secondary reactions at typical operating conditions, which prevents one from equating their production rates with the primary rate of oxidative scission. In contrast, the carboxylic acids initially formed through oxidative scission are stable, and their production rates are used to define mass-normalized rates of methyl and alkyl scission (Eq. 1 and Eq. 2):

$$\begin{matrix}{R}_{M} = \frac{{F}_{{C}_{( n - 1 )} OOH}}{{m}_{c}} & ( 1 ) \\ {R}_{A} = \frac{{F}_{EA}}{{m}_{c}} & ( 2 ) \\ {S}_{M} = \frac{{R}_{M}}{{R}_{M} + {R}_{A}} & ( 3 ) \\ {S}_{A} = \frac{{R}_{A}}{{R}_{M} + {R}_{A}} & ( 4 )\end{matrix}$$

Methyl scission produces carboxylic acid fragments that vary with the structure and chain length of the probe molecule. The rate of methyl scission (RM) is thus uniquely defined for each probe molecule as the effluent molar flowrate of the Cn-1 carboxylic acid divided by the mass of catalyst (mC) in the packed bed reactor (Eq. 1), where n is the total number of carbon atoms in the probe molecule. Conversely, alkyl scission of a methylketone always produces ethanoic acid (acetic acid) as a primary product; accordingly, the rate of alkyl scission (RA) for all species is given by the effluent flowrate of ethanoic acid (FEA). As shown in Eq. 3 and Eq. 4, rates of methyl and alkyl scission are used to define selectivities toward methyl (SM) and alkyl scission (SA). Oxidative C—C scission is irreversible under our reaction conditions; therefore, scission selectivity is kinetically controlled. Except for 2-propanone (acetone), all probe molecules are asymmetric, and carboxylic acids formed through methyl and alkyl scission pathways are easily resolved by gas chromatography.

Trends in the oxidative scission of linear and branched methylketones at 468K are summarized in Table 1 below.

TABLE 1
Oxidative scission rates and selectivities toward methyl
and alkyl scission for linear and branched methylketones.

| | | Oxidative Scission Rate | | | | |
|---|---|---|---|---|---|---|
| | | (μmol g⁻¹min⁻¹) | | | Selectivity (%) | |
| Entry | Substrate | Me | Alk | Total | Me | Alk |
| 1 | 2-propanone | 1.7 | N/A | 3.4 | 100 | N/A |
| 2 | 2-butanone | 0.5 | 12.5 | 13 | 4 | 96 |
| 3 | 2-pentanone | 0.5 | 7.9 | 8.4 | 5 | 95 |
| 4 | 2-hexanone | 0.3 | 7.4 | 7.7 | 4 | 96 |
| 5 | 2-heptanone | 0.3 | 6.8 | 7.1 | 4 | 96 |
| 6 | 2-octanone | 0.3 | 7.1 | 7.4 | 4 | 96 |
| 7 | 3-Me-2-P | 0.5 | 40 | 40 | 1 | 99 |
| 8 | 3,3-diMe-2-P | 0.3 | 0 | 0.3 | 100 | 0 |
| 9 | 4-Me-2-P | 0.7 | 6.2 | 6.9 | 11 | 89 |
| 10 | 4,4-diMe-2-P | 1.0 | 2.0 | 3.0 | 34 | 66 |

Methyl (Me); Alkyl (Alk); 2-pentanone (2-P).
T = 468 K, P_{Ketone} = 12.3 Torr; P_{O2} = 133 Torr; Helium balance.

All rates were obtained over VOx/Al2O3 under differential conditions. 2-propanone is a linear, symmetric ketone with indistinguishable methyl groups in each α-position. Scission of either α-carbon forms ethanoic acid, which is produced at a rate of 3.4 μmol g⁻¹ min⁻¹. Assuming that each α-carbon contributes half to the total ethanoic acid production rate, it is possible to infer an intrinsic methyl scission rate of 1.7 μmol g⁻¹ min⁻¹. In contrast, C4-C8 methylketones are asymmetric, permitting independent quantification of methyl and alkyl scission rates. A threefold decrease was observed in methyl scission rate upon transitioning from 2-propanone (1.7 μmol g⁻¹min⁻¹) to 2-butanone (0.5 μmol g⁻¹ min⁻¹). Thereafter, rates of methyl scission are comparable for 2-pentanone (0.5 μmol g⁻¹ min⁻¹), 2-hexanone (0.3 μmol g-1 min⁻¹), 2-heptanone (0.3 μmol g⁻¹ min⁻¹), and 2-octanone (0.3 mol g⁻¹ min⁻¹). In total, methyl scission rates decrease significantly between 2-propanone (C3) and 2-butanone (C4) and then become insensitive to chain length in the C4 -C8 range, where a mean rate of 0.38±0.3 μmol g⁻¹ min⁻¹ (95% confidence interval) was observed. Relative to the methyl scission rate established for 2-propanone (1.7 μmol g⁻¹ min⁻¹), extending the chain by a single carbon (2-butanone) increases the rate of alkyl scission by an order-of-magnitude (12.5 μmol g⁻¹ min⁻¹). Subsequent chain extension to 2-pentanone decreases the alkyl scission rate to 7.9μmol g⁻¹ min⁻¹, whereas further chain extension only slightly reduces alkyl scission rates for 2-hexanone (7.4 μmol g⁻¹ min⁻¹), 2-heptanone (6.8 μmol g⁻¹ min⁻¹), and 2-octanone (7.1 μmol g⁻¹ min⁻¹). The mean alkyl scission rate for C5-C8 methyl ketones is 7.3±1.5 μmol g⁻¹ min⁻¹ (95% confidence interval), and only 2-butanone falls outside this range, suggesting that alkyl scission rates, like methyl scission rates, initially decrease with chain length but become insensitive in the C5-C8 range.

From the delineation of methyl and alkyl scission rates, three important observations may be highlighted. First, transitioning from methyl scission (2-propanone) to alkyl scission (2-butanone) confers an order-of-magnitude increase in oxidation rate. This implies that a secondary α-carbon is far more susceptible to oxidation than a primary α-carbon. Second, within a given category of α-carbons (i.e., considering the scission of either primary or secondary α-carbons), oxidative cleavage rates initially show a strong, inverse scaling with chain length (C3-C5) and thereafter are insensitive to the size of the molecule (≥C5). Alkyl chain length is not expected to significantly influence free energies of activation or reaction for the elementary scission and bond formation steps that comprise ketone oxidation, so this effect is tentatively attributed to reduced coverages for increasingly bulky probe molecules. This has not been confirmed by measuring adsorption isotherms; however, it is reasonable to expect that repulsive lateral interactions become significant at lower coverages for larger molecules and that this impact is more pronounced when extending a short chain ketone (e.g., 2-propanone vs. 2-butanone) than when extending a long chain ketone (e.g., 2-heptanone vs. 2-octanone). Finally, rates of alkyl scission are always roughly an order of magnitude higher than rates of methyl scission. This results in an alkyl scission selectivity of ≈95%, which is independent of the probe molecule and aligns with the preference for alkyl scission that is typically reported over vanadium oxides.

The impact of branching and carbon substitution in the α- and β-positions of the alkyl side chain was considered next. Adding a methyl substituent to the 3-position of 2-pentanone, creates a tertiary α-carbon in the alkyl side chain. This increases the rate of alkyl scission five-fold relative to that observed for linear C5-C8 methylketones (40 μmol g⁻¹ min⁻¹ vs. 7.3±1.8 μmol g⁻¹ min⁻¹). On the other hand, the rate of methyl scission for 3-methyl-2-pentanone is indistinguishable from methyl scission rates in C4-C8 linear methylketones (0.5 μmol g⁻¹ min⁻¹ vs. 0.38±0.3 μmol g⁻¹ min⁻¹). An enhanced alkyl scission rate alongside a static methyl scission rate improves alkyl scission selectivity to 99% for 3-methyl-2-pentanone. Dimethyl substitution at the α-position of 2-pentanone (3,3-dimethyl-2-pentanone) creates a quaternary α-carbon. After rigorous subtraction of trace ethanoic acid associated with feed impurities (3,3-dimethyl-2-pentanone was available at a purity of 96%), no evidence that 3,3-dimethyl2-pentanone undergoes alkyl scission was found, so it was concluded that oxidative cleavage is completely suppressed for a quaternary α-carbon. In contrast, its rate of methyl scission is identical to the average observed for C4-C8 linear methylketones (0.3 μmol g⁻¹ min⁻¹ vs. 0.38±0.3 μmol g⁻¹ min⁻¹). Since no alkyl scission is observed, 3,3-dimethyl-2-pentanone oxidation is 100% selective toward methyl scission. To probe the impact of substitution at the (β-position, the oxidative scission of 4-methyl-2-pentanone and 4,4-dimethyl-2-pentanone was considered. Data reveal a monotonic increase in methyl scission selectivity with increasing substitution at the (β-carbon. Specifically, methyl scission selectivities of 5% for 2-pentanone, 10% for 4-methyl-2-pentanone and 34% for 4,4-dimethyl-2-pentanone were observed. The shift in selectivity arises from a simultaneous increase in methyl scission rates and suppression of alkyl scission rates. This suggests that steric restrictions play a role in dictating oxidative cleavage selectivity.

The rate of oxidative scission increases monotonically with degree of substitution for primary, secondary, and tertiary α-carbons, yet scission is entirely suppressed for a quaternary α-carbon. For oxidative scission to occur, it is clear that the α-carbon must be bound to at least one hydrogen atom. This stands to reason as, macroscopically, oxidative scission of methylketones converts an sp³ α-carbon (e.g., CH₃) into an sp² carbonyl (e.g., HCHO), which requires elimination of an α-hydrogen. Because fragmentation is completely suppressed for a quaternary α-carbon, the scission of the α-CH bond must mechanistically precede scission of the α-CC bond. Finally, the observation that rates are enhanced by carbon substitution indicates that the rate controlling step involves either formation or consumption of an intermediate that is stabilized by increased electron density at the α-carbon, e.g., an α-radical or a α-cation (or, in this case, their surface analogs). These conclusions align with mechanisms that have been previously suggested for ketone oxidation in solution. First, whether they are acid-, base-, or radical-initiated, homogeneous ketone oxidations proceed through a sequence of α-hydrogen elimination followed by α-CC scission. Second, they all ultimately involve homolytic reactions that form a radical at the α-carbon. This radical reacts with dioxygen to form either a peroxy or a hydroperoxy species, which subsequently attacks the electrophilic carbonyl carbon to drive C—C scission.

α-hydrogen elimination clearly plays an important role in ketone oxidation over VOx/γ-Al₂O₃.Further, one expects that the rate of α-CH scission will be sensitive to the degree of α-carbon substitution. This sensitivity may underlie our observation that ketone oxidation rates depend on the degree of α-carbon substitution, so it is worth considering whether α-CH scission is rate controlling. The relationship between the kinetics of α-CH scission and the degree of α-carbon substitution is dictated by the nature of C-13 H scission, which can occur in one of three ways: homolytic bond scission to form a hydrogen atom and a carbon radical; heterolytic deprotonation to form a proton and a carbanion; and heterolytic hydride elimination to form a hydrogen anion and a carbocation. VOx/γ-Al₂O₃ purportedly has surface acid, base, and redox functionality; and it can facilitate Brønsted and Lewis interactions as well as single electron oxidations. Thus, it can potentially catalyze any C-13 H scission pathway, and we accordingly consider their kinetic significance.

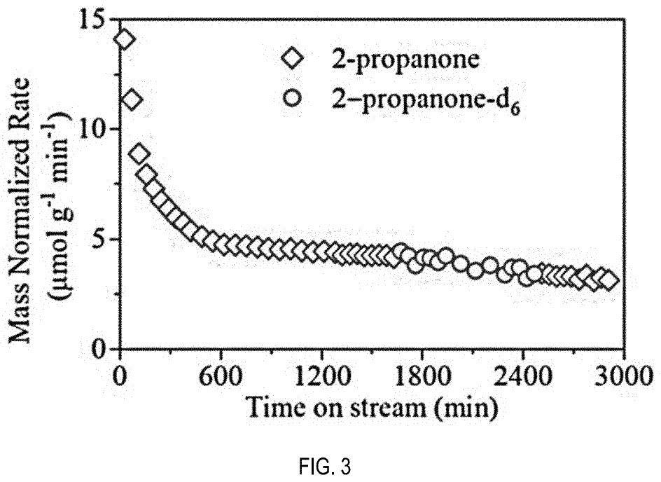

α-hydrogen deprotonation is possible and perhaps even likely on an amphoteric solid oxide; however, alkyl scission rates that increase with α-carbon substitution argue against rate control by deprotonation of the α-hydrogen. The pKa for a C-13 H bond increases with the degree of carbon substitution. pKa is taken as a proxy for C-13 H deprotonation energies, so it is possible to infer that these will also increase with carbon substitution. Within a given class of reactions, kinetic barriers scale linearly with reaction enthalpies; thus, deprotonation barriers should increase with pKa, and deprotonation rates should decrease as observed during aldol condensation in alkaline media. For this reason, it may be concluded that if α-CH scission is rate controlling, it must either occur through homolytic scission to form an α-radical or hydride elimination to form an α-carbenium (or surface analogs thereof). C-13 H bond dissociation energies decrease with carbon substitution, and proton affinities for alkenes (negative enthalpies of protonation) increase with carbon substitution. Again, assuming that kinetic barriers scale linearly with reaction enthalpies, one expects that rates of both homolytic C-13 H scission and heterolytic hydride elimination will increase with carbon substitution; thus, rate control by either would be consistent with the observations. To further interrogate the kinetic significance of α-CH scission, rates of oxidative scission for 2-propanone and perdeuterated 2-propanone (FIG. 3) are compared, and it may be clearly observed that deuterium substitution at the α-carbon has no impact on the rate of oxidative scission.

The lack of a primary kinetic isotope effect indicates that, although it is mechanistically required to precede α-CC scission, α-CH scission is likely not kinetically significant. Though less definitive, the lack of a secondary kinetic isotope effect³⁸⁻⁴⁰ may argue that the rate determining step does not involve the α-carbon at all—this despite our observation that increasing substitution at the α-carbon enhances the overall rate of oxidation. One rationale is that α-CH scission appears to precede α-CC scission; thus the α-carbon undergoing oxidation is initially hydrogen deficient and may be loosely described as having sp² hybridization. As it undergoes the α-CO bond formation and/or α-CC bond scission steps necessary to effect oxidative cleavage and ultimately form a carbonyl, one might anticipate transitions between sp² and sp^{a} hybridization at the α-carbon. It is therefore not unreasonable to expect a modest secondary kinetic isotope effect if either α-CC scission or α-CO bond formation are rate controlling. Additional work is necessary to fully resolve rate control in this system, but based on a clear lack of kinetic isotope effects upon deuterium substitution at the α-carbon, the observation that oxidative scission rates increase with a carbon substitution may be tentatively attributed to a thermodynamic effect. Most likely, the rate controlling step in ketone oxidation simply involves consumption of a species whose equilibrium bulk and/or surface concentrations are enhanced by increasing substitution at the α-carbon.

Having mapped reactivity and selectivity trends during the oxidative scission of monofunctional ketones, levulinic acid oxidation was considered next. Levulinic acid is far less susceptible to oxidative scission than its monofunctional analogs (Table 1, 468K); accordingly, its oxidation was carried out at 550K, and observations benchmarked against 2-pentanone oxidation at the same temperature (Table 2):

TABLE 2
Rates and selectivities observed during the oxidative scission
of 2-pentanone, levulinic acid, and α-angelicalactone.

| | | Oxidative Scission Rate | | | | |
|---|---|---|---|---|---|---|
| | | (μmol g⁻¹min⁻¹) | | | Selectivity (%) | |
| Entry | Substrate | Me | Alk | Total | Me | Alk |
| 1 | 2-P | 7 | 140 | 147 | 5 | 95 |
| 2 | LA | 30 | 11 | 41 | 73 | 27 |
| 3 | α-AL | 29 | 5 | 34 | 85 | 15 |

Methyl (Me); Alkyl (Alk); 2-pentanone (2-P); levulinic acid (LA); α-angelicalactone (α-AL).
T = 550 K, P_{Ketone} = 12.3 Torr; P_{O2} = 133 Torr; Helium balance.

Levulinic acid reacts by other pathways at this temperature, so conversions here are not strictly differential (see subsequent contact time analysis). The total oxidative scission rate for 2-pentanone increases from 8.4 μmol g⁻¹ min⁻¹ at 468K (Table 1) to 147 μmol g⁻¹ min⁻¹ at 550K (Table 2); however, selectivities toward alkyl scission (95%) and methyl scission (5%) are identical to those observed at lower temperatures. Transitioning to levulinic acid, the rate of methyl scission (30 μmol g⁻¹ min⁻¹) is significantly higher than that observed for 2-pentanone (7 μmol g⁻¹ min⁻¹), whereas the rate of alkyl scission is profoundly diminished (11 μmol g⁻¹ min⁻¹ vs. 140 μmol g⁻1 min⁻¹). This gives rise to alkyl and methyl scission selectivities of 27% and 73%, respectively. Based on data in Table 1, this is difficult to reconcile with the fact that levulinic acid has a secondary α-carbon in its alkyl side chain. With no other structural differences, perturbations in the rates of both methyl scission (5-fold increase) and alkyl scission (10-fold decrease) are clearly attributable to the presence of the carboxyl group in levulinic acid, so possible origins were explored next.

The analysis of monofunctional ketone oxidation (Table 1) indicates that steric hindrances at the alkyl chain α-carbon can improve methyl scission selectivity by simultaneously enhancing methyl scission rates and suppressing alkyl scission rates. That said, the impact is relatively minor, and a significant steric effect—an increase in methyl scission selectivity from 5% to 34%—was only observed in the case of a bulky dimethyl substituent at the β-position (i.e., immediately adjacent to the α-carbon). It thus seems unlikely that a carboxyl group at the y-position (relative to the ketone) could impose sufficient steric hindrances to shift selectivity so strongly toward methyl scission. Alternatively, it was been demonstrated that rates of oxidative scission increase with electron density (alkyl substitution) at the α-carbon. One may propose that the carboxyl group of levulinic acid draws electron density away from the alkyl α-carbon and consequently suppresses its rate of scission; however, one expects a relatively weak impact since any such induction effects are screened at the α-carbon by an intervening methylene group. It thus seems unlikely that induction effects would make alkyl scission less favorable than methyl scission in levulinic acid. A final possibility is that the carboxyl group enables formation of unique bulk and/or surface structures that are less susceptible to alkyl scission and/or more susceptible to methyl scission than analogous monofunctional ketones.

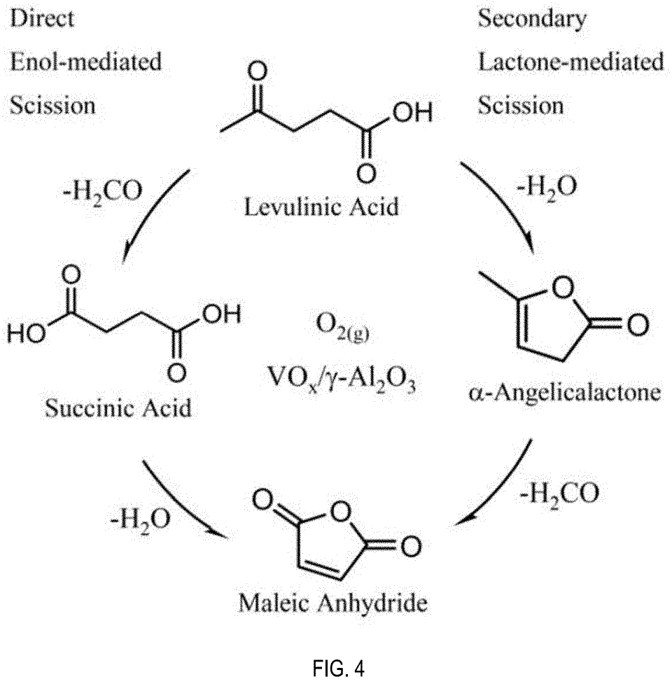

Oxidative ketone scission must occur through open chain, enol-mediated pathways, which are commonly reported for ketone oxidations in solution and on reducible oxide surfaces. If levulinic acid oxidation follows an open chain pathway, then its dominant oxidation product, maleic anhydride, must form through a sequence of methyl scission (to form succinic acid) followed by dehydration and oxidative dehydrogenation (FIG. 4).

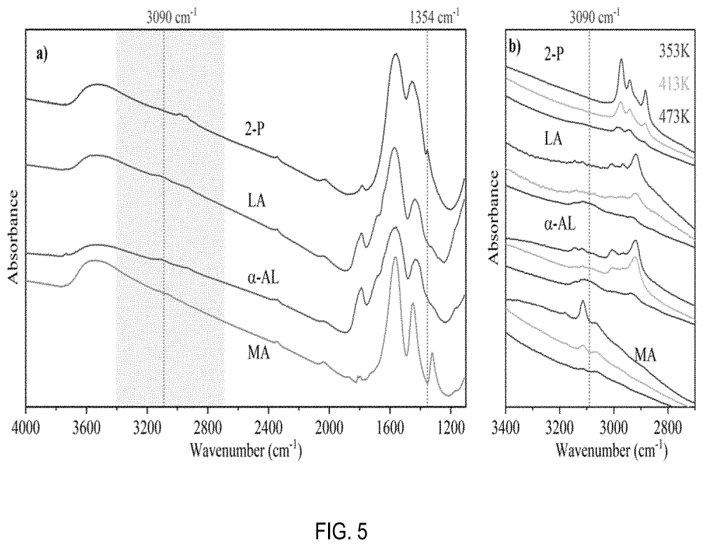

It is worth considering whether the cyclization and oxidative scission steps actually happen in reverse. Specifically, levulinic acid will readily undergo intramolecular dehydration form cyclic angelicalactone isomers (α, α′, and β). Levulinic acid dehydration is both kinetically facile and thermodynamically favorable under our reaction conditions, making it plausible that dehydration precedes oxidative scission in this system. Accordingly, the rate of oxidative scission for α-angelicalactone was measured at identical conditions to levulinic acid and 2-pentanone (Table 2). At 29 μmol g⁻¹ min⁻¹, the methyl scission rate for α-angelicalactone is nearly identical to that observed for levulinic acid and significantly higher than that observed for 2-pentanone. In contrast, its alkyl scission rate (5 μmol g⁻¹ min⁻¹) is further suppressed relative to levulinic acid, causing a small increase in methyl scission selectivity (85%). These results suggest that levulinic acid and α-angelicalactone proceed through common oxidative scission pathways and that these pathways may be distinct from those governing 2-pentanone oxidation. This hypothesis was further probed using in situ transmission FTIR spectroscopy, which provides insight into the nature of surface species formed upon exposing VOx/y-Al₂O₃ to 2-pentanone (2-P), levulinic acid (LA), α-angelicalactone (α-AL), and maleic anhydride (MA) under O2 (FIG. 5).

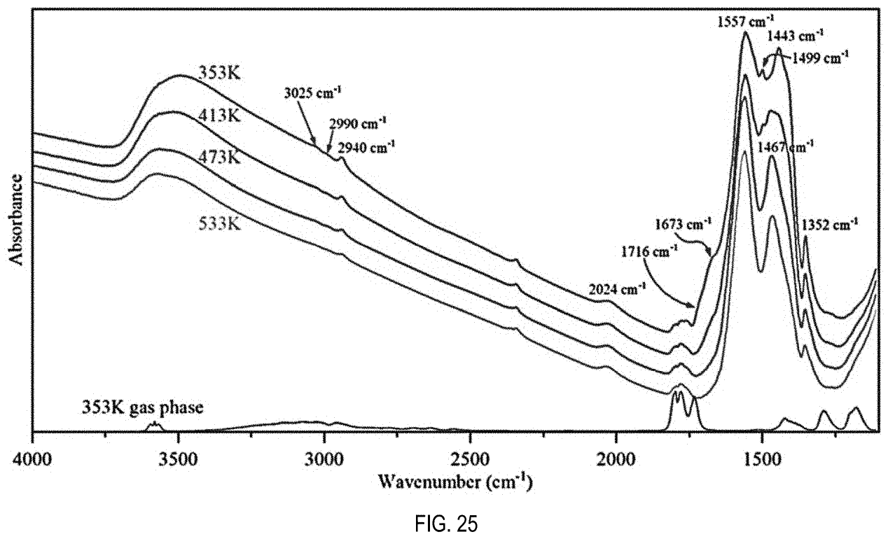

At 473K, full-scale spectra for α-angelicalactone and levulinic acid are nearly identical (FIG. 5a), indicating that common surface intermediates form during the oxidation of both molecules. Further, comparison with the 2-pentanone spectrum suggests that these intermediates are different from those formed during oxidation of a monofunctional ketone. At 473K, the 2-pentanone spectrum is dominated by carboxylate (O—C—O) stretching modes (1454 and 1564 cm⁻¹) and a CH deformation mode (1354 cm⁻¹).⁴⁵⁻⁴⁷ Based on a comparison with reference spectra (FIG. 25), these bands are all assigned to surface-bound acetate. This is consistent with the facile production of ethanoic acid via alkyl scission of 2-pentanone, which occurs readily at 468K (Table 1). Analogous spectra for levulinic acid and α-angelicalactone also show carboxylate signatures, however, comparison with a reference spectrum indicates that these features are distinct from their dominant oxidative scission product, adsorbed maleic anhydride (FIG. 5a). This aligns with the observation that levulinic acid and α-angelicalactone do not undergo appreciable oxidative scission below ≈500K. Accordingly, it may be suggested that these bands are associated with distinct surface intermediates that mechanistically precede the oxidative scission of α-CC bonds.

The region between 2800 and 3300 cm⁻¹, where various C-13 H stretching modes that change with the identity of the probe molecule and the reaction temperature (FIG. 5b) were observed, is highlighted. In the case of 2-pentanone, alkyl C-13 H stretching modes (2800-3000 cm⁻¹) dominate at all reaction temperatures. In contrast, during the oxidation of levulinic acid and α-angelicalactone, it was observed that both alkyl C-13 H stretching modes (2800-3000 cm⁻¹) and alkenyl C-13 H stretching modes (3050-3200 cm⁻¹). At low temperatures, alkyl and alkenyl C-13 H bands co-exist, which is consistent with chemisorbed angelicalactones or their open-chain analogs (enol tautomers of levulinic acid). As temperatures increase, the alkyl C-13 H bands disappear almost entirely, and the spectra become dominated by alkenyl C-13 H bands. The alkenyl bands observed during the oxidation of levulinic acid and a -angelicalactone change qualitatively (and in a similar way) as a function of reaction temperature. Both species display the same alkenyl bands at low temperatures (3143 cm⁻¹ and 3116 cm⁻¹, 353K). Subsequently, the bands shift and intensify, ultimately manifesting as a broad feature at 3090 cm' (473K). The similarity of the bands indicate that levulinic acid and α-angelicalactone likely form the same surface intermediates under oxidizing conditions. Further, the loss of alkyl C-13 H stretching modes alongside increasingly prominent alkenyl features indicates that these surface intermediates are hydrogen-deficient relative to both levulinic acid and α-angelicalactone. These observations are potentially consistent with the formation of their dominant oxidation product, maleic anhydride, which has no sp³ carbons and thus no alkyl C-13 H bonds; however, comparison with reference spectra (FIG. 5b) indicates that these new alkenyl C-13 H bands are distinct from the relatively well-defined alkenyl C-13 H stretching modes observed for chemisorbed maleic anhydride. Again, this is consistent with the observation that neither levulinic acid nor α-angelicalactone undergo appreciable oxidative scission below roughly 500K; accordingly, it may be concluded that, during the oxidation of levulinic acid and a -angelicalactone, dehydrogenation reactions form increasingly hydrogen-deficient surface species prior to the onset of oxidative scission reactions that form maleic anhydride.

Reaction networks may be further investigated by analyzing trends in carbon selectivity as a function of contact time during the oxidation of levulinic acid and α-angelicalactone. Before doing so, carbon balance closure and observed reaction products are discussed. During oxidation of both species, the presence of maleic anhydride, α-angelicalactone, 3-angelicalactone, ethanoic acid, CO, CO2, and methyl vinyl ketone was confirmed by comparison of retention times and/or GCMS fragmentation patterns with reference standards. GCMS analysis also indicated the formation of two additional species. The first had a total molecular mass of 98 (C₅H₆O₂) and a fragmentation pattern that matched that of 1,3-cyclopentanedione, while the other had a total molecular mass of 96 (C₅H₄O₂) and a fragmentation pattern that matched 4-cyclopentene-1,3-dione. Detailed analysis of the fragmentation pattern for the 98 m/z species suggests that its structure is far more consistent with α′-angelicalactone, which was confirmed by matching its retention time and fragmentation pattern with that of a commercial standard. A detailed analysis of the fragmentation pattern for the 96 m/z species was inconclusive, and it was not possible to further unable to match its retention time with a commercial standard of its suggested match (4-cyclopentene-1,3-dione). Accordingly, NMR was used to determine its structure.

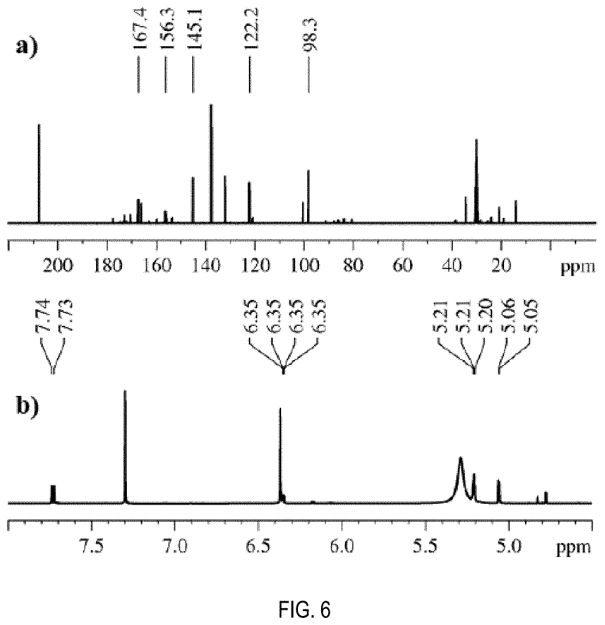

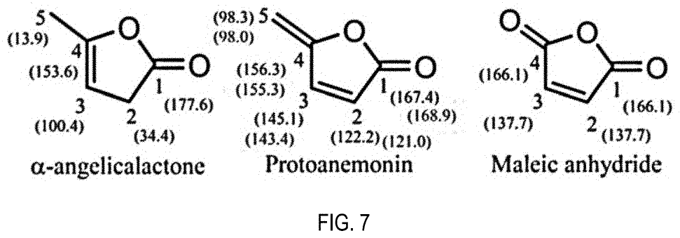

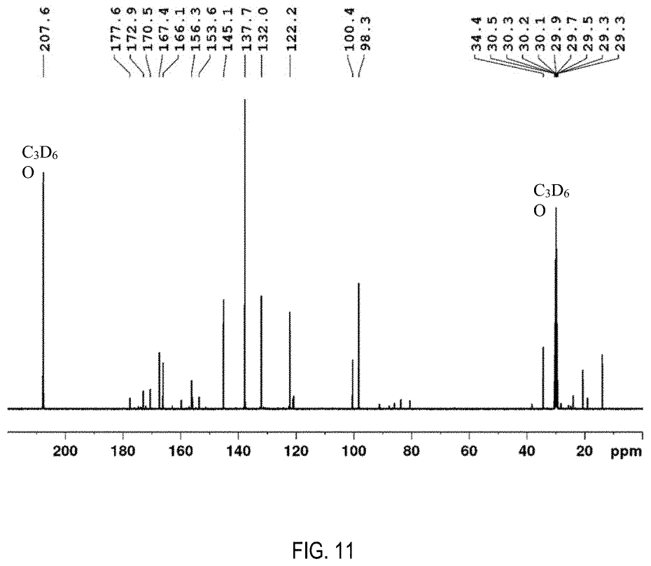

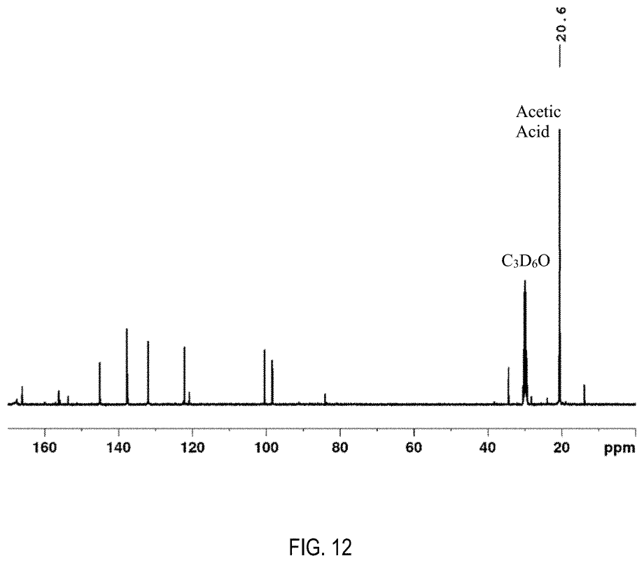

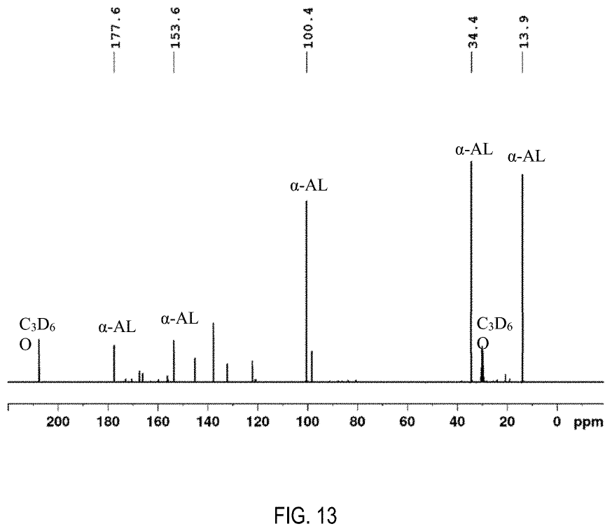

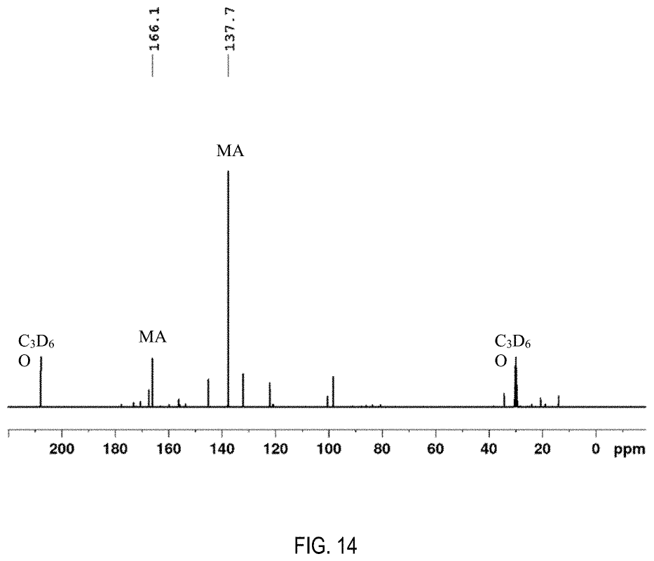

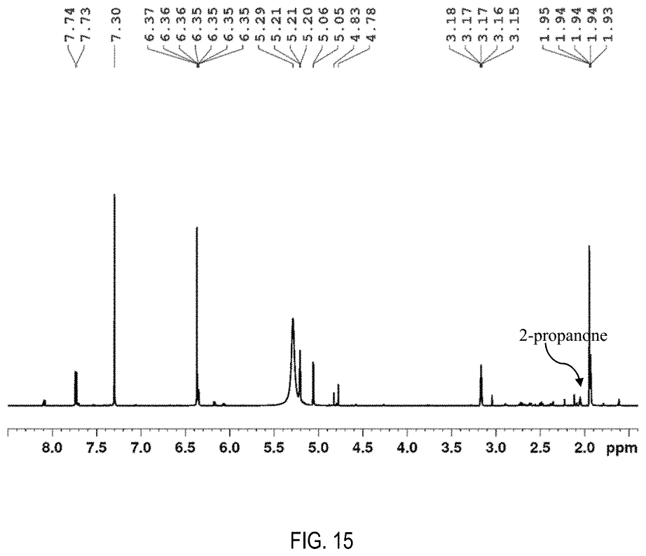

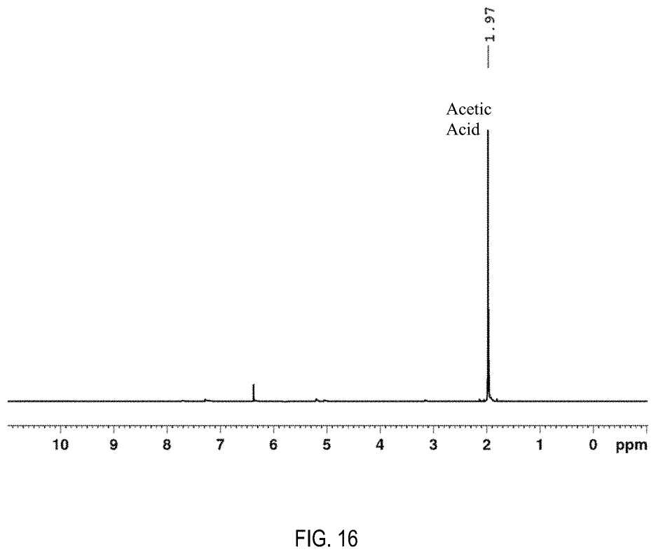

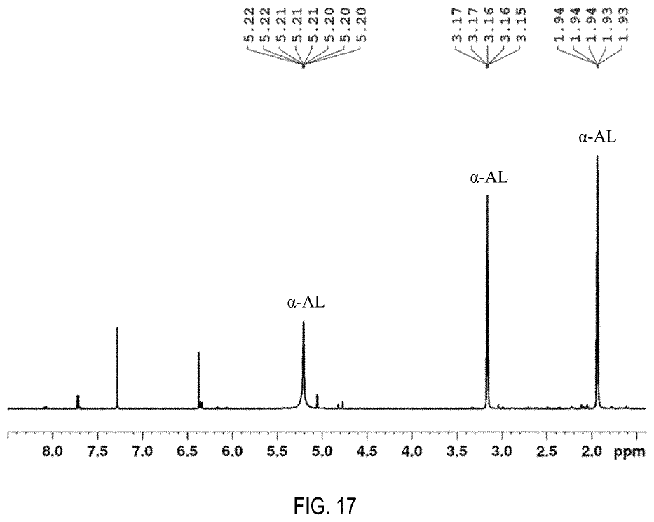

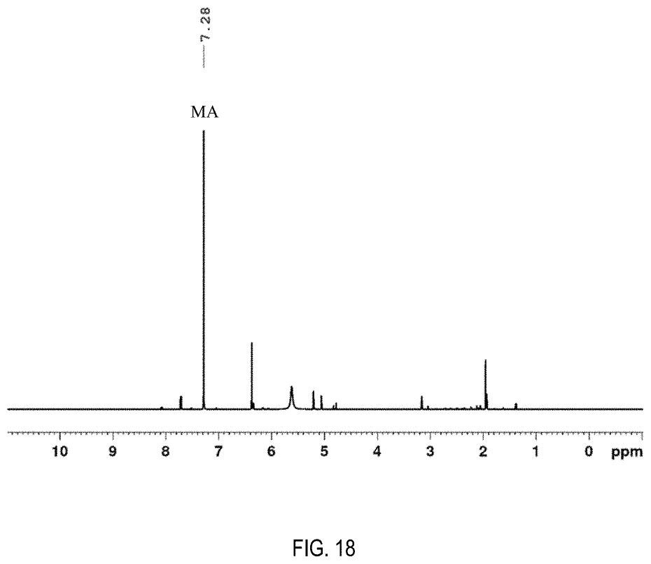

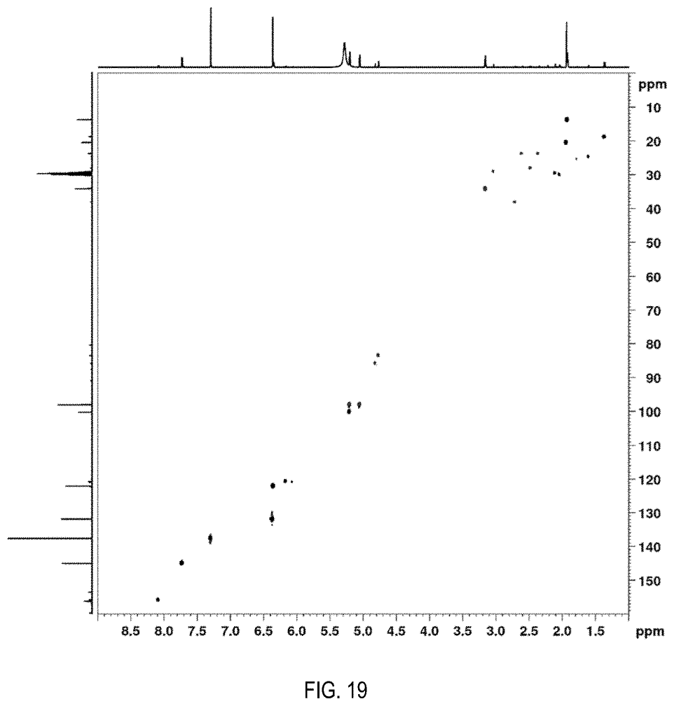

FIG. 6 presents ¹³C- NMR and ¹H-NMR for a product mixture obtained from α-angelicalactone oxidation under conditions that were selective toward the unidentified 96 m/z species. Its associated peaks are labelled by their chemical shift; they were assigned by elimination. All reference and full range spectra are provided in the online supporting information (FIGS. 11-18). The ¹³C- spectrum contains five peaks assigned to the unknown compound (FIG. 6a). Based on comparisons with reference spectra and general correlations between chemical shift and carbon environment, the peaks at 98.3, 122.2, 145.1, and 156.3 ppm are assigned to 4 distinct carbon atoms present in C═C double bonds, and the peak at 167.4 ppm is assigned to a carbon atom in an acid anhydride or lactone structure. In the ¹H-NMR spectrum (FIG. 6b), 4 distinct hydrogen atoms that give rise to peaks (5.05, 5.21, 6.35 and 7.74 ppm) with varying degrees of splitting were observed. Most likely, these are attributable to four distinct alkenyl hydrogens. These assignments are reinforced by the ¹H-—¹³C HSQC spectrum (FIG. 19), which indicates that two hydrogens (5.05 ppm and 5.21 ppm) are bound to a primary alkene carbon (98.3 ppm), while two additional hydrogens (6.35 ppm and 7.74 ppm) are bound to distinct secondary alkene carbons (122.2 and 145.1 ppm) in an oxolene group. Considering these structural insights alongside a total molecular mass of 96 amu (GCMS) and the indication from FTIR that levulinic acid and α-angelicalactone form increasingly hydrogen-deficient intermediates under reaction conditions, it is possible to conclude that the unknown is the dehydrogenated analog of β-angelicalactone, specifically, 5-methylidenefuran-2-one (protoanemonin). A simulated 13C spectrum for protoanemonin was generated using ChemDraw 18.0, and it agrees well with the proposed assignments (FIG. 7).

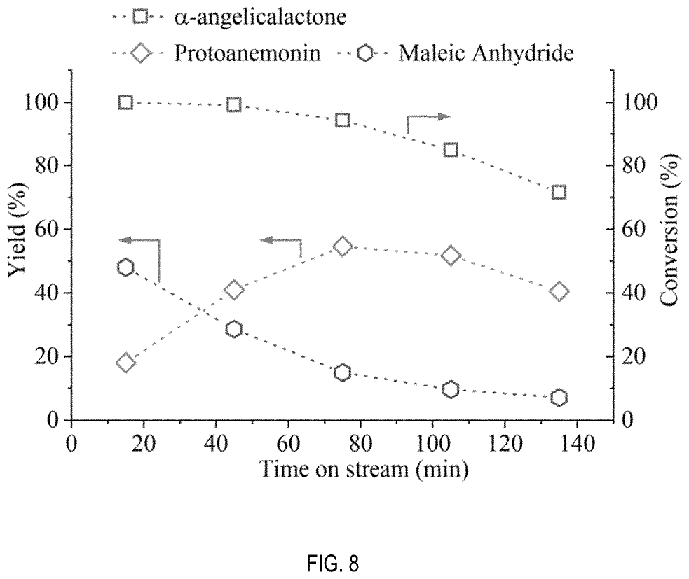

Before considering the role of protoanemonin during levulinic acid oxidation, we note that protoanemonin is a polyfunctional molecule comprised of a lactone ring alongside a conjugated diene. Protoanemonin has previously been discussed in the plant science and medical literature, where there was some historical interest in its antimicrobial properties. Relevant to future biorefining opportunities, dienes and lactones are routinely leveraged in polymer chemistry, making protoanemonin a unique and intriguing platform for bio-based polymers. To date, only one account of protoanemonin synthesis has been reported using levulinic acid as a starting material (with one other account describing its synthesis from β-acetylacrylic acid, i.e., dehydrogenated levulinic acid). It was accomplished in good yield using a conventional organic methodology, namely through a sequence of acid-catalyzed reactive distillation of levulinic acid to form α-angelicalactone followed by bromination and dehydrobromination of α-angelicalactone to form protoanemonin. Here, attempts at yield optimization were limited to enriching protoanemonin concentration for the benefit NMR analysis; however, even with this cursory effort, the present invention achieved transient protoanemonin yields between 55% and 75% via simple, direct, gas-phase oxidation of α-angelicalactone (e.g., see FIG. 8). These yields are sufficiently high to suggest potential scalability (though our operating concentrations are presently low). The reaction is not thermodynamically limited, which motivates future efforts at improving selectivity via catalyst and process design. Attempts to isolate and purify our dilute protoanemonin product (<10 Torr) generally induced polymerization, and the lack of a commercial standard for protoanemonin prevented a precise GC-FID response factor from being obtained. It was thus assumed that protoanemonin has the same HD response factor as its isomer, 4-cyclopentene-1,3-dione (C₅H₄O₂). Carbon balances closed to within 10%, suggesting this is a reasonable approximation; however, there is uncertainty in our reported yields and production rates.

The evolution of product selectivity was analyzed as a function of contact time during levulinic acid oxidation. Consideration was not restricted to oxidative scission, and selectivities were calculated for all products on a carbon basis (SC,]) as shown in Eq. (6). Here F] is the molar flowrate of species], NC] is the number of carbon atoms in a molecule of species], and the index “]” excludes the reacting molecule.

$${S}_{C , j} = \frac{{F}_{j} {N}_{C , j}}{{\sum }_{j} {F}_{j} {N}_{C , j}} \qquad ( 6 )$$

The discussion focuses on cyclic structures—angelicalactones (α, α′, and β), protoanemonin, and maleic anhydride, and the remaining products are excluded for clarity. Presented carbon selectivities do not necessarily sum to 1; however, carbon balances fully closed for each experiment.

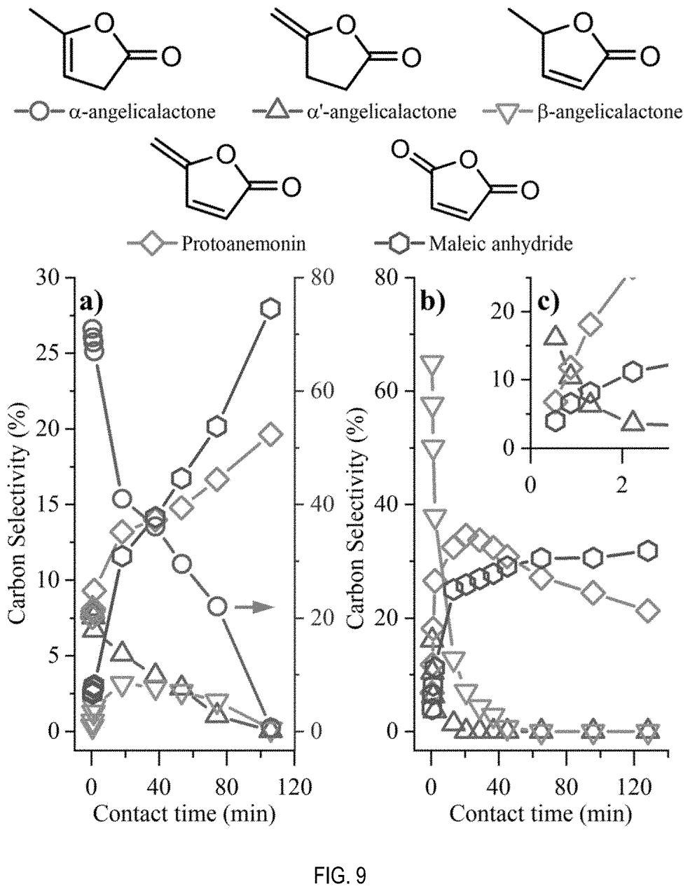

FIG. 9a shows that, during levulinic acid oxidation, only two products, α-angelicalactone and α′-angelicalactone, have positive y-intercepts. These translate to non-zero selectivities at the zero conversion limit, which indicates that both α-angelicalactone and α′-angelicalactone are formed through reactions that directly consume levulinic acid. Selectivities toward both α- and α′-angelicalactone decrease as a function of contact time, which implies that they are each consumed by secondary reactions. In contrast, β-angelicalactone selectivity extrapolates to zero at the y-intercept, indicating that it is not a primary product of levulinic acid conversion. β-angelicalactone selectivity then increases to a maximum at a contact time of 20 minutes before diminishing at longer contact times. Its selectivity increase mirrors the decrease in selectivity toward α- and α′-angelicalactones, which indicates that β-angelicalactone forms through reactions that consume the α-isomers. The decay in β-angelicalactone selectivity indicates that it is further converted through tertiary pathways. During levulinic acid oxidation, selectivities to protoanemonin and maleic anhydride both extrapolate to a zero y-intercept and subsequently increase alongside the decrease in β-angelicalactone selectivity. This indicates that both protoanemonin and maleic anhydride form through tertiary pathways via consumption of β-angelicalactone; however, their relationship is unclear based on analysis of levulinic acid oxidation selectivity alone. To aid in resolution, the analogous selectivity trends observed during α-angelicalactone oxidation (FIG. 9b) were considered. In this case, both α′ and β-angelicalactone are primary products (positive y-intercept) that form directly by consumption of α-angelicalactone. β-angelicalactone selectivity decreases with contact time, indicating subsequent consumption by secondary reactions. Protoanemonin has a zero y-intercept, and its selectivity increases with contact time alongside a decrease in β-angelicalactone selectivity. It may therefore be concluded that protoanemonin forms as a secondary product of reactions that consume β-angelicalactone. Subsequently, protoanemonin selectivity decreases, indicating that it is converted by a tertiary pathway. Maleic anhydride selectivity also extrapolates to a zero y-intercept (i.e., it is not a primary product). Further, its selectivity increases with the onset of protoanemonin formation and continues to increase alongside the decay in protoanemonin selectivity at higher contact times. This strongly suggests that maleic anhydride forms through a tertiary reaction that consumes protoanemonin directly. Finally, the selectivity plateau observed for maleic anhydride suggests that it is a stable product under our experimental conditions.

Based on the above delineation of primary and sequential reaction products, it is proposed that, during levulinic acid oxidation, maleic anhydride forms through the network shown in Scheme 3. Initially, levulinic acid dehydrates to form α- and α′-angelicalactones. Subsequent isomerization forms β-angelicalactone, which undergoes oxidative dehydrogenation at its C4-05 bond to form protoanemonin. Protoanemonin has a single C═C unsaturation adjacent to the ring oxgyen, which is similar to the enol-type structures that are widely thought to precede oxidative scission of ketone α-carbons; accordingly, it is proposed that maleic anhydride forms through oxidative scission of the C4═C5 bond in protoanemonin. If one follows the logic that enol-like structures must precede oxidative C—C scission, then protoanemonin, once formed, is susceptible only to scission of the C4=C5 bond. This corresponds to the bond between the carbonyl carbon and its a-methyl carbon in both levulinic acid and α-angelicalactone, i.e., the one that is cleaved in order to form maleic anhydride. Intermediate formation of protoanemonin therefore provides a plausible explanation for suppressed alkyl scission rates and the corresponding high selectivity toward methyl scission observed during the oxidation of levulinic acid and α-angelicalactone.

A limited oxidation susceptibility of protoanemonin is reasonable, and it explains why protoanemonin should have a high selectivity toward methyl scission; however, it does not immediately explain why the oxidation of levulinic acid and angelicalactone are so selective toward methyl scission. In either case, protoanemonin formation must proceed through α′- or α-angelicalactone. Both α-isomers have C═C bonds adjacent to the ring oxygen, which, following the rationale presented for protoanemonin, should make them susceptible to oxidative scission, yet no succinic anhydride (scission of C4=C5 bond in the α′-isomer) and very little acetate/ethanoic acid (scission of the C3=C4 bond in the α-isomer) was observed during the oxidation of either levulinic acid or α-angelicalactone. The lack of succinic acid can be reasonably attributed to the fact that the α′-isomer is thermodynamically unfavorable and generally present in low concentrations; however, the α-isomer is favorable, and it was frequently observed that product distributions that are rich in α-angelicalactone. Considering this alongside the apparent oxidation susceptibility of the C3=C4 bond in α-angelicalactone, it is not immediately clear why alkyl scission rates approach zero (Table 2) during the oxidation of both levulinic acid and α-angelicalactone. The answer lies in the relative rates and equilibrium positions for dehydration, isomerization, oxidative dehydrogenation, and oxidative scission.

Under our typical operating conditions, levulinic acid is nearly equilibrated with a- and a′-angelicalactones, indicating that its dehdryation occurs rapidly. The migration of C=C bonds is also facile over solid oxides, which explains why it was observed that angelicalactone distributions that are rich in the thermodynamically preferred isomers (α- and β-)instead of only those formed directly by dehydration (α- and α′-). Subsequent oxidative dehydrogenation of β-angelicalactone forms protoanemonin and water, which is thermodynamically favorable and may be considered irreversible. If levulinic acid dehydration and C═C bond migration are facile, it is reasonable to assume that any β-angelicalactone consumed by oxidative dehydrogenation is rapidly offset by isomerization of α/α′-isomers and further dehydration of levulinic acid. In a system where levulinic acid dehydration, C═C bond migration, and oxidative dehydrogenation are facile (but oxidative scission is not kinetically accessible), one expects a high concentration of the thermodynamic product, which is protoanemonin. Once formed, protoanemonin cannot revert to β-angelicalactone, and it is susceptible only to oxidative scission at the C4=C5 bond; thus, the balance between methyl scission and alkyl scission should be dictated by the relative rates of oxidative scission of the C3=C4 bond in α-angelicalactone and oxidative dehydrogenation of the C4-05 bond in β-angelicalactone. If oxidative dehydrogenation of β-angelicalactone is rapid relative to oxidative scission of α-angelicalactone, one will preferentially form protoanemonin and observe a high methyl scission selectivity. If the converse is true, one should observe appreciable selectivity to acetate/ethanoic acid via C3=C4 scission of α-angelicalactone.

To test this hypothesis, α-angelicalactone and 2-pentanone was fed into the oxidation reactor at identical conditions (T =473 K, Pprobe molecule=10 Torr, PO2=133 Torr). During the oxidation of α-angelicalactone, an initial protoanemonin formation rate of 442 μmol g⁻¹min⁻¹ was observed with no evolution of maleic anhydride. Protoanemonin is a secondary product in this system, and this production rate was measured at 92% conversion of α-angelicalactone and 75% yield of protoanemonin, i.e., at near-complete angelicalactone conversion (all isomers). 442 μmol g⁻¹ min⁻¹ thus reflects an average rate of oxidative dehydrogenation over the catalyst bed, and taken as a lower limit on the intrinsic rate of oxidative dehydrogenation of β-angelicalactone at the reactor inlet. At identical conditions, 2-pentanone undergoes oxidative alkyl scission at a rate of 118 μmol g⁻¹min⁻¹, which was measured at 16% conversion. This production rate reasonably approximates a differential rate of reaction, and taken it as an upper limit on the rate of oxidative scission for a secondary α-carbon at the reactor inlet. The rate of alkyl scission in 2-pentanone is a reasonable proxy for the expected rate of oxidative scission for the C3=C4 bond in α-angelicalactone since both have a secondary α-carbon. It may therefore be concluded that oxidative dehydrogenation of the C4-05 bond in β-angelicalactone can be reasonably expected to occur roughly 4-times more quickly than the oxidative scission of a secondary α-carbon. If, as speculated, the balance between methyl scission and alkyl scission is determined by the ratio of the rate of oxidative dehydrogenation of β-angelicalactone to the oxidative scission of a secondary α-carbon, one expects methyl scission and alkyl scission will be observed in a roughly 4:1 ratio. This aligns remarkably well with the observed selectivities during the oxidative scission of levulinic acid (73% methyl vs. 27% alkyl) and α-angelicalactone (85% methyl vs. 15% methyl) at 550K.

The influences of ketone structure and secondary function on rates and selectivity during oxidative scission over VOx/γ-Al₂O₃ were investigated. Oxidative scission reactions are moderately sensitive to steric effects, and it was observed that α-hydrogen elimination is a necessary precursor to oxidative scission of the α-CC bond. This criteria can be met for primary, secondary, and tertiary α-carbons, for which oxidative scission rates increase with degree of the α-carbon substitution. Interestingly, neither primary nor secondary kinetic isotope effects were observed during the oxidative scission of 2-propanone and 2-propanone-d6, which may argue that the rate determining step in ketone oxidation does not involve formation or scission of a bond at the α-carbon (at least not on VOx/γ-Al₂O₃).

Similarities in selectivity and surface intermediates formed during the oxidation of levulinic acid and α-angelicalactone suggest the two species undergo a common oxidation pathway that is distinct from that of a typical methylketone. Specifically, both molecules appear to undergo dehydrogenation reactions that form identical surface intermediates prior to oxidative C—C scission behavior that is not observed during 2-pentanone oxidation. A detailed analysis of product selectivities at very short contact times reveals that levulinic acid rapidly dehydrates to form α, α′, and β-angelicalatones, which are thermodynamically favored relative to levulinic acid under our reaction conditions. β-angelicalactone undergoes irreversible oxidative dehydrogenation to form protoanemonin, which was conclusively identified using NMR spectroscopy. Once formed, protoanemonin is only susceptible to oxidation via scission of its primary α-carbon, i.e., the C4=C5 bond, to form maleic anhydride. Importantly, oxidative dehydrogenation of β-angelicalactone is sufficiently fast to limit the extent of oxidative scission of the C3=C4 bond in α-angelicalactone, which minimizes selectivity toward alkyl scission. Ultimately, the high yield of maleic anhydride during levulinic acid oxidation is attributed to a kinetically and thermodynamically favorable cascade of reactions that occur quickly relative to the oxidative scission of an α-carbon. This cascade leads to protoanemonin, which forms irreversibly and is susceptible only to oxidation via scission of its primary α-carbon.

As a final note, the attempt to resolve the origin of maleic anhydride selectivity lead us to isolate protoanemonin via gas-phase oxidation of levulinic acid and α-angelicalactone. Protoanemonin has lactone and diene functionality, and it is an interesting platform for the synthesis of bio-polymers. Its unique functionality may offer performance advantages relative to conventional petrochemical monomers. This is the first reported synthesis of protoanemonin using heterogeneously-catalyzed, gas-phase, aerobic oxidation of two relatively accessible bio-based commodities: levulinic acid and α-angelicalactone. Further, in a flow process, yields in the range of 50%-75% have been observed during periods of transient operation. Protoanemonin yields are not thermodynamically limited, suggesting potential feasibility and motivating further optimization of angelicalactone dehydrogenation.

Supporting Information

1. Materials and Methods

1.1 Reagents

Rates and selectivities during the oxidative scission of 2-propanone (Acros, 99%), 2-propanone-d6 (Aldrich, 99.9% D), 2-butanone (Acros 99%), 2-pentanone (Acros, 99%), 2-hexanone (Acros 98%), 2-heptanone (Acros, 98%), 2-octanone (Acros, 99%), 3-methyl-2-pentanone (Sigma 97%), 4-methyl-2-pentanone (Sigma, 99%), 3,3-dimethyl-2-pentanone (Oakwood, 96%), 4,4-dimethyl-2-pentanone (Acros, 99%), levulinic acid (Acros, 98%), and α-angelicalactone (Alfa Aesar, 98%). Additional reference samples were used for species identification and instrument calibration. These include ethanoic acid (Acros, 98%), propanoic acid (Acros 99%), butanoic acid (Acros 99%), pentanoic acid (Acros, 99%), hexanoic acid (Acros, 99%), heptanoic acid (98%, Acros), 2-methyl butanoic acid (Sigma, 98%), 2,2-dimethyl butanoic acid (Acros, 99%), 3,3-dimethyl butanoic acid (Sigma, 98%), maleic anhydride (TCI, 99%), 1,3-cyclopentanedione (Acros, 99%), 4-cyclopentene-1,3-dione (Aldrich, 95%), ethanal (Sigma, >99.5%), propanal (Acros, 99%), butanal (Acros, 99%), pentanal (Acros, 98%), hexanal (Alpha Aesar, 98%), a′-angelicalactone (TCI, 98%), CO (Airgas, 1%, 1% Ar, He balance) and CO2 (Airgas, 1%, 1% Ar, He balance). Our catalyst synthesis method used γ-Al₂O₃ (Strem, 95%), ammonium metavanadate (Sigma, 99.5%), and oxalic acid (Acros, 99%). β-angelicalactone was not available commercially, so it was synthesized by mixing triethylamine (Acros, 99%) with α-angelicalactone in 1:1 molar ratio and stirring at room temperature for 16 hours. This resulted in a two-phase mixture with a dense lower phase containing angelicalactone isomers in a molar ratio of 2:8 (α:β). It was extracted and used to acquire FTIR reference spectra, calibrate GC retention times, and obtain reference MS fragmentation patterns. For quantitative analysis, the HD response factor for β-angelicalactone was taken to be equal to that of its isomer, α-angelicalactone. Water was purified by reverse osmosis, UV oxidation, and ion exchange to a resistivity>18.2 MΩ′cm (Spectrapure). O2 (Airgas, Ultra High Purity) and He (Airgas, Ultra High Purity) were used as oxidant and diluent during reactor operation. Air (Airgas, Ultra Zero) was used for ex-situ calcination of catalyst samples.

1.2 Catalyst Synthesis

Experiments were carried out using a VOₓ/γ-Al₂O₃ catalyst prepared at a loading of 8.0 V nm⁻² by impregnation of aqueous vanadium oxalate onto a γ-Al₂O₃ support (231 m2 g-1) that was pre-calcined under zero-grade air (723K, 3 K min⁻¹ ramp, 4h hold, 60 ml min ⁻¹). Ammonium metavanadate and oxalic acid were dissolved in water at 343K at 1:2 molar ratio. The solution was then added to the calcined γ-Al₂O₃ at a loading of 1.1 mL solution per gram of γ-Al₂O₃. The resulting slurry was dried in an oven at 338 K for 16 hours and then calcined under flowing air (723K, 3 K min⁻¹ ramp, 4h hold, 60 ml min^{-I}). The catalyst was then crushed and sieved. All samples utilized here had a particle size range of 45-90 μm.

1.3 Oxidative Scission Reactions

Rates and selectivities for oxidative scission were measured at steady state in a packed bed reactor. 50-100 mg of VOx/γ-Al₂O₃ were diluted to 20 wt.% in quartz granules (45-90 μm) and placed between quartz wool plugs at the center of a 6″ long ½″ OD stainless steel tube. The tube upstream of the catalyst bed was filled with quartz chips, and an inline type-K thermocouple was positioned in the void space downstream of the catalyst bed to monitor the internal temperature. All transfer lines were heat traced and held at 500K, which was sufficient to prevent condensation and/or crystallization of all species at their representative partial pressures. Effluent concentrations were measured using an inline gas chromatograph (HP 5890). It was equipped with two inlets, two columns, and two detectors. An HP-INNOWax column was paired with an FID detector for analysis of hydrocarbons, and we used a Restek ShinCarbon ST Micropacked column paired with a TCD detector for analysis of carbon oxides. Reaction products were qualitatively identified by matching GC retention times with those of reference standards, by comparison with NIST libraries using GC-MS (Agilent 7890+5975C MSD), and/or by solution NMR (Bruker Avance III HD 400 MHz). Carbon balances for all experiments closed to ≥95%, and monitoring of internal and external bed temperatures confirmed isothermal operation.

VOx/γ-Al₂O₃ deactivates during oxidative scission, and reactors typically reach steady state within 20 hours on stream. Unfortunately, the rate and extent of deactivation vary with the identity of the probe molecule, which demands caution when comparing steady-state rate measurements between different probe molecules. To address this, each experiment was initialized with 2-pentanone oxidation (466K or 550K, WHSV=1.95 h⁻¹, 12.3 Torr 2-pentanone, 133 Torr O₂, He balance). The system was allowed to reach steady state, and effluent flowrates were quantified. Subsequently, a step change was made in probe molecule identity while holding all other parameters constant. The system was again allowed to reach steady state, and effluent flowrates were again quantified. Finally, the probe molecule was switched back to 2-pentanone, and the system was permitted to return to steady state. Doing so allowed us to correct for catalyst deactivation and/or induction caused by a change in probe molecule identity, ensuring a common basis for comparing oxidation rates for varying probe molecules. Deactivation modes were not characterized, but catalyst activity was fully restored by calcination in He/O2 mixture (133 Torr O2, He balance) at 450° C., implicating either carbon deposition or reduction of the lattice. Upon regeneration, catalyst beds retained their initial reference activity indefinitely (>10 cycles).

1.4 FTIR Spectroscopy

In situ transmission FTIR was employed to probe the nature of surface species during ketone oxidation (Nicolet 6700, DTGS detector). We used a home built-cell constructed from a vacuum tee (McMaster-Carr, 1½″ OD) with electrical feedthroughs and CaF₂ windows. The cell was configured to accommodate an aluminum sample holder positioned orthogonal to the IR beam. The sample holder was drilled to accept two cartridge heaters (McMaster, ⅛×1″×1 ¼″) and a type K thermocouple, which were used with a PID controller (Love Controls, Series 16A) to regulate temperature. To allow gas flow or sample evacuation, two ¼ tubes were welded onto the vacuum tee body; each was equipped with a ¼ bellows valve. Prior to introducing the catalyst sample, the cell was purged under continuous air flow (Peak Scientific, PG28L), and the sample holder was heated to 723K (3 K min⁻¹, 4h hold, 100 ml min⁻¹). The sample holder was then cooled to 573K and purged for 30 minutes with a He/02 blend (Airgas, 15% 0₂, 85% He, 100 ml min⁻¹) that was purified by passing sequentially through a liquid nitrogen trap and a molecular sieve trap (Agilent, MT200-4). FTIR spectra were then acquired at 5 minute intervals until the system reached steady state. The final spectrum was used as the background reference, and the sample holder was cooled to ambient temperature. Subsequently, 15 mg of sample (VOₓ/γ-Al₂O₃ or γ-Al₂O₃) were pressed into a wafer using a hydraulic press (Specac M26855), secured in the aluminum sample holder, and subjected to an identical calcination procedure to that described for the empty cell. Reference spectra of the calcined catalyst and support were obtained at each reaction temperature. Probe molecules were next introduced by vaporizing species into the He/02 carrier at a pressure of 0.2 Torr, which proved sufficiently high to generate detectable coverages of surface species and sufficiently low to minimize gas-phase bands and avoid condensate formation. Probe molecules were introduced through capillary tubing into a temperature-controlled vaporizer comprised of a stainless-steel tee that was heat-traced, insulated, and packed with quartz wool. Liquid flow was controlled using a syringe pump (Cole Parmer series 100). Maleic anhydride is a high-melting solid, so it was introduced by sublimation. Specifically, the carrier was passed through a temperature-controlled saturation chamber containing solid maleic anhydride. After exposing the catalyst to the probe molecule, FTIR spectra were acquired at 5 minute intervals until reaching steady state. The sample temperature was then increased by 60 K (3K/min), and the process was repeated until reaching 473K. Both the cell body and transfer lines were heat-traced and held at sufficient temperatures to prevent condensation of probe molecules and their oxidation products at representative partial pressures.

1.5 NMR Spectroscopy

NMR was used to determine the structure of a previously unidentified reaction intermediate observed during the oxidation of levulinic acid and α-angelicalactone. NMR spectra were recorded at 300 K on Bruker AVANCE III HD 400MHz spectrometer equipped with a liquid nitrogen-cooled Prodigy probe. NMR samples were prepared by bubbling vapor-phase reaction products through a cold trap containing perdeuterated 2-propanone (2-propanone-d6, C₃D₆O ). Attempts to isolate and purify the unknown reaction product induced polymerization and precipitation, which prevented a detailed structural assignment. It was thus necessary to record spectra for unrefined reaction products. The mixture was enriched in the target molecule by operating reactors under conditions that gave high selectivity to the unknown intermediate (confirmed by GC). Reference spectra were obtained for all known species present in appreciable concentrations in the product mixture. These were ethanoic acid, α-angelicalactone, and maleic anhydride. Each was separately dissolved in an aliquot of the C₃D₆O solution containing reaction products, which permitted resolution of peaks associated with known compounds from those associated with the unidentified intermediate. 1D 1H spectra were obtained at a spectral width of 20 ppm in 16 scans with a relaxation delay of 1s and 65536 data points. Proton spectra were referenced to the 2-propanone methyl hydrogen peak at 2.05 ppm. 1D 13C spectra were acquired at a spectral width of 239 ppm in 256 scans with a relaxation delay of 2s and 65536 data points. Carbon spectra were referenced to the 2-propanone methyl carbon at 29.93 ppm. ¹H¹³C HSQC spectra were collected using the Bruker pulse sequence hsqcedetgpsisp 2.3, which includes multiplicity editing during the selection step. HSQC spectra had 2048×256 data points with spectral widths of 13 ppm in the ¹H dimension and 165 ppm in the ¹³C- dimensions. 4 scans were acquired during each t1 increment. HSQC data were apodized by a cos 2 function in both dimensions with zero-filling in the remote dimension to give a final data set of 1000×1000 points. Spectra were analyzed using Bruker's Topspin 3.6.1 software. Qualitative peak assignments were made using comparisons to reference standards and, where necessary, predicted NMR spectra generated using ChemDraw 18.0.

1.6 Contact Time Experiments

To distinguish between primary and sequential reaction products during the oxidative scission of levulinic acid and α-angelicalactone, product selectivities were measured as a function of contact time (τ), which is defined per Eq. 5 as the catalyst mass (mC) divided by the influent mass flowrate of the probe molecule (mR).

$$τ = \frac{{m}_{C}}{{\dot{m}}_{R}} \qquad ( 5 )$$

Contact time experiments were performed at 550K under 5 Torr of levulinic acid or α-angelicalactone and 133 Torr 02 in a He balance. To separate lactone isomers, a Restek RTX-1701 column was used in GC-FID described in Section 2.3; otherwise, the reactor and analytical equipment were identical to the previously described system (Section 1.3). Each experiment was initialized by feeding the probe molecule into the reactor and allowing it to reach steady state. Subsequently, the contact time was varied by changing the inlet flowrates of all species as necessary to maintain constant feed partial pressures, and the system was again permitted to reach steady state. It was observed that contact time had no significant impact on catalyst deactivation; accordingly, contact times were varied progressively without returning to initial conditions, and deactivation corrections were not employed. This approach is acceptable since the probe molecule identity remained fixed for the duration of the experiment, and the evolution of selectivity was discussed only as a function of contact time rather than making a comparison of rates as a function of time on stream. Where it was necessary to increase or decrease contact time beyond the range permitted by modulation of inlet flowrates, the mass of catalyst was varied.

What is claimed is:

1. A process of forming protoanemonin, comprising the steps of:
providing an amount of levulinic acid or an amount of α-Angelica lactone; and
performing oxidative dehydrogenation of the amount of levulinic acid or the amount of α-Angelica lactone to form an amount of protoanemonin.

2. The process of claim 1, wherein the step of oxidative dehydrogenation is heterogeneously catalyzed.

3. The process of claim 1, wherein the step of oxidative dehydrogenation is performed in a gas-phase.

4. The process of claim 1, wherein the step of oxidative dehydrogenation comprises aerobic oxidation.

5. The process of claim 1, wherein the step of oxidative dehydrogenation is performed with a solid oxide catalyst.

6. The process of claim 5, wherein the solid oxide catalyst is performed with vanadium oxide.

7. The process of claim 1, wherein the step of oxidative dehydrogenation includes the formation of β-Angelica lactone as an intermediate product.

8. The process of claim 1, wherein the step of oxidative dehydrogenation is performed at a temperature of 473 Kelvin.
